(19) United States
(12) Patent Application Publication
(10) Pub. No.: US 2021/0298263 A1
(43) Pub. Date: Sep. 30, 2021

(54) New Citrus Variety 'Jedae mandarin'

(71) Applicant: JEJU NATIONAL UNIVERSITY INDUSTRY-ACADEMIC COOPERATION FOUNDATION, Jeju-si, Jeju-do (KR)

(72) Inventors: In-Jung KIM, Jeju-si, Jeju-do (KR); Ji-Man HEO, Jeju-si, Jeju-do (KR)

(73) Assignee: JEJU NATIONAL UNIVERSITY INDUSTRY-ACADEMIC COOPERATION FOUNDATION, Jeju-si, Jeju-do (KR)

(21) Appl. No.: 15/740,806

(22) PCT Filed: Jun. 29, 2016
(86) PCT No.: PCT/KR2016/006967

§ 371 Date: May 3, 2018

(30) Foreign Application Priority Data
Jun. 29, 2015 (KR) 10-2015-0092516
Jun. 29, 2016 (KR) 10-2016-0081748

(51) Int. Cl. A01H 6/78; A01H 5/08

(52) U.S. Cl. CPC A01H 6/785; A01H 5/08

(57) ABSTRACT

The present disclosure relates to ‘Jedae mandarin’, a new citrus variety of early-ripening satsuma mandarin. The branch buds of C. unshiu Marc. cv. Miyagawa-wase, an early-maturing satsuma mandarin, underwent gamma irradiation and subsequent grafting for asexual propagation. As a result, the ‘Jedae mandarin’, a novel variety of citrus plant, showed uneven peels of fruits and changes in flavonoid content, compared to C. unshiu Marc. cv. Miyagawa-wase, although it exhibited characteristics very similar to those of C. unshiu Marc. cv. Miyagawa-wase in terms of blooming, fruit-bearing, sugar content, acidity, etc. Thus, ‘Jedae mandarin’ may be used as a new variety with high economic value.

### TECHNICAL FIELD

The present invention relates to ‘Jedae mandarin’, a new citrus variety of early-ripening satsuma mandarin with altered fruit shape and flavonoid content.

### BACKGROUND ART

Citrus is one of the most prominent fruit crops in Korea, taking top or second spot in terms of production value nationwide, depending on production year. However, location of its production is limited to Jeju, unlike other fruit crops.

The early-ripening satsuma mandarin is a variety that can be cultivated when the annual average temperature is equal to or exceeds 15° C., the lowest temperature for commercially cultivated citrus varieties, and the minimum temperature in winter is −7° C. or higher. The early-ripening satsuma mandarins are difficult to be cultivated in sub-tropical regions where the minimum temperature is high in winter during which coloration is not properly achieved, resulting in plain taste. The United States and other Western countries are aiming to develop mandarin-based varieties characterized by seedlessness, thin peel and ease of peeling, like early-ripening satsuma mandarin, in connection with breeding of mandarins.

C. unshiu Marc. cv. Miyagawa-wase of early-maturing satsuma mandarin is the most cultivated varieties in Jeju Island. This variety, growing naturally in Fukuoka Prefecture in Japan, was found to have branches mutated from those of ordinary satsuma mandarin of indigenous varieties and became a cultivated variety in 1925. The line of this variety was introduced into Korea and cultivated as one of the most prominent varieties of early-maturing satsuma mandarin. C. unshiu marc. var. okitsu, the second most widely cultivated citrus, is a variety resulting from mutation of branches of C. unshiu Marc. cv. Miyagawa-wase.

The cultivation area of early-ripening satsuma mandarin, including C. unshiu Marc. cv. Miyagawa-wase, is very high, comprising more than 90% of total citrus cultivation area (Statistics Korea, KREI Agricultural Outlook for 2015). Most of produced citrus fruits are used for reproductive purposes, and some of non-commercial citrus fruits out of the range of standard sizes are used for processing the commodities such as juice, etc.

As a result, it is necessary to develop varieties that have wide-ranging applications such as ornamental purposes and processing purposes with greater functionality, as well as reproduction purposes.

Although early-ripening satsuma mandarin does not create seeds, the seeds of early-ripening satsuma mandarin can be obtained through artificial cross-breeding with mother plants that create seeds. As the seeds are multi-breeding and pollen is hardly formed, cultivation of new varieties by cross-breeding is very difficult. In addition, it is unlikely that varieties with better traits will be developed than those being currently cultivated.

As a result, studies have been conducted vigorously to investigate the mutation breeding that involves exposure to radioactive sources mainly in the United States, Israel, and Japan (US PP 08378; Nucleus., 2000, 37(1-2):107-112).). Based on results of studies on such radiation mutation breeding, varieties with traits such as seedlessness, color variation, and easy peeling have been developed and distributed to farming sites.

‘Radiation mutation breeding’ technology refers to the technology used to induce mutations of genes or chromosomes through irradiation of radiation to plant seeds or seedlings and then to select mutants with excellent traits in descendant generations and to develop new genetic resources through genetic fixing process.

It is a breeding technique that raises the incidence of mutations occurring at low frequencies in the natural state through radiation stimulation. Radiation mutation breeding technology has proven safety, unlike genetically modified technology which artificially inserts foreign genes, and is used to improve food crops such as rice and soybeans and to develop new varieties of flowers and fruits.

### PRECEDING TECHNICAL LITERATURES

(Patent Literature 0001) Korea PP 10-0532072, published on Nov. 29, 2005
(Patent Literature 0002) US PP08378
(Non-Patent Literature 0001) Nucleus., 2000, 37(1-2):107-112

### DISCLOSURE

### Technical Problem

The objective of the present invention is to provide a variant of citrus plant named ‘Jedae mandarin’, an early-maturing satsuma mandarin characterized by fruit shape and flavonoid components altered by artificial mutation breeding method involving the gamma irradiation.

### Technical Solution

To accomplish aforesaid objective, the method for breeding a variant of citrus plant, which is a new variety named ‘Jedae mandarin’ bred in accordance with the embodiment of the present invention, involves the following stages: 1) a stage in which buds of branches of C. unshiu Marc. cv. Miyagawa-wase are irradiated with gamma rays; 2) a stage in which buds, irradiated with gamma rays as described in the foregoing stage 1), are grafted to induce branching and thereafter the buds are collected from mutant branches that have fruits with altered shape; and 3) a stage in which the mutant branches' buds, selected as described in the foregoing stage 2), are grafted onto the rootstocks.

Jedae mandarin bred in that way is an early-maturing satsuma mandarin with these characteristics: i) rough peel, ii) vertical corrugation from fruit stalk which results in uneven outer appearance of fruit, and iii) higher flavonoid content in the fruit, compared to flavonoid content in the fruit of C. unshiu Marc. cv. Miyagawa-wase, and asexual reproduction and propagation.

The new variety citrus plant, named ‘Jedae mandarin’ in accordance with an embodiment of the present invention, is a mutant plant bred from C. unshiu Marc. cv. Miyagawa-wase and is an early-maturing satsuma mandarin reproduced and propagated asexually, and furthermore, characterized by i) rough peel, ii) vertical corrugation from fruit stalk which results in uneven outer appearance of fruit, and iii) higher flavonoid content in the fruit, compared to flavonoid content in the fruit of C. unshiu Marc. cv. Miyagawa-wase.

The citrus, as specified by another embodiment of the present invention, is a fruit of a novel variant of citrus plant named ‘Jedae mandarin’, harvested from a variant of citrus plant named ‘Jedae mandarin’ described above.

Regarding the asexual breeding method of the variant of citrus plant named ‘Jedae mandarin’ in accordance with another embodiment of the present invention, the variant of citrus plant named ‘Jedae mandarin’ can be bred and propagated asexually with the process of grafting the buds of the variant of citrus plant named ‘Jedae mandarin’ onto the rootstocks.

The term, ‘early-maturing satsuma mandarin’, used in the present invention refers to the citrus that can be harvested earlier. Citrus unshiu Marc. cv. Miysagawa-wase of the present invention has been bred since satsuma mandarin, a conventional plant type growing naturally in Fukuoka Prefecture of Japan, was found to have mutant branches. It is one of the most typical citrus varieties of early-maturing satsuma mandarin, which has been also cultivated commonly in Korea.

The term ‘grafting’ in the present invention refers to an artificial cultivation technique for cutting parts of same or different plants and joining them to grow one. It is a method for attaching the buds or stems with buds(scion, upper part of graft) to the roots or stems with roots(rootstock, lower part of graft) to produce grafted seedlings. They are adhered to one another and connected to vascular bundles and joining bundles by callus created through grafting in the formative layer of rootstock and scion, resulting in growth into full plant. Grafting is classified into the category of branch grafting, bud grafting, succulent grafting, etc., depending on whether the scion is the branch, bud or shoot. Branching grafting classified into subcategory of cut grafting, cleft grafting, side grafting, top grafting, etc., depending on the method for cutting and joining the branches on rootstocks. Grafting is not limited to aforesaid methods. In the present invention, however, it is preferable to graft the parts through top grafting or cut grafting, and specific grafting methods can be carried out by conventional methods well known to those who are skilled in relevant fields.

As the grafting methods are not limited to aforesaid methods, the mature tree of C. unshiu Marc. cv. Miyagawa-wase, the contrasting type of the present invention, can be used as rootstock, as well as the trifoliate orange tree used as rootstock of citrus tree.

The term ‘asexual propagation’ in the present invention means the method such as unsexual reproduction, in which a single individual forms a new individual without the need for male and female. Asexual propagation refers to the case where reproductive cells produced by an individual form a new individual by itself. Asexual reproduction refers to all breeding methods other than seed propagation(sexual propagation). Although propagation method is not limited to asexual reproduction, it is preferable to adopt asexual propagation method through grafting in the present invention.

The present inventors applied mutation breeding technology to C. unshiu Marc. cv. Miyagawa-wase of early-maturing satsuma mandarin, the variety cultivated most extensively. As a result, a novel variety of citrus plant named ‘early-maturing satsuma mandarin’ with altered fruit shape and fruit content, was developed, thus bringing the present invention to completion.

Specifically, the present inventors sought to develop high-quality early-ripening satsuma mandarin varieties aiming at diversification of citrus varieties. As a result, mutation breeding technology was adopted which involved radiation irradiation to C. unshiu Marc. cv. Miyagawa-wase of early-maturing satsuma mandarin, the citrus plant cultivated most extensively in Korea to an extent that it has the largest cultivation area among crop plants across the country. Based on that technology, a new citrus variety of early-maturing satsuma mandarin with altered fruit shape and flavonoid component was bred, thus bringing the present invention to completion.

The present invention provides a method for breeding and asexual propagation of a variant of citrus plant named ‘Jedae mandarin’ and citrus and citrus plants, which has altered fruit shape and flavonoid component.

Description in the present specification, which shows the characteristics of the variant of citrus plant, such as the weight, size, peel thickness, sugar content, acidity, peel color of fruit, and extent of development of vascular bundles in fruit skin, etc., as well as vitality of the plant, should be interpreted in view of the fact that some changes may occur in the characteristics of the plant within a range of acceptable change due to the nature of plants themselves or plants reproduced asexually. That is not interpreted to mean unreasonably limiting the characteristics of a variant of citrus plants named ‘Jedae mandarin’ described in this Specification, and consequently the scope of the appended claims is not limited.

When the variant of citrus plant named ‘Jedae mandarin’ of the present invention is cultivated by its breeding method or asexual propagation method, etc., there may be some difference in terms of characteristics of the fruit, such as fruit weight, size, fruit skin thickness, sugar content, acidity, fruit skin color, fruit shape, content of functional ingredients, etc., as well as some difference in the plant itself such as vitality of tree, depending on cultivation conditions such as cultivation region, cultivation location, climate, soil, weather, and house or non-house cultivation. Moreover, some change may be found in the characteristics of the fruit, depending on the age of plant.

When aforesaid difference in characteristics of the fruit may be attributed to external factors other than the characteristics of the breed itself, and therefore may be interpreted as changes in characteristics which arise from environmental changes in the same plant variety, such difference is considered to be covered by the scope of appended claims.

The description for the embodiment of a variant of citrus plant named ‘Jedae mandarin’ of the present invention is based on the cultivation in open fields of Jeju Special Self-Governing Province of Korea where the breeding was performed, unless otherwise specified.

For achievement of aforesaid objective, the fruit of a variant of citrus plant named ‘Jedae mandarin’ in accordance with an embodiment of the present invention is a type of early-maturing satsuma mandarin and has the following characteristics: i) rough peel, ii) uneven outer appearance of fruit, and iii) higher flavonoid content in the fruit, compared to flavonoid content in the fruit of C. unshiu Marc. cv. Miyagawa-wase.

The fruits of aforesaid ‘Jedae mandarin’ has characteristics similar to those of C. unshiu Marc. cv. Miyagawa-wase in terms of iv) vitality of tree, blooming period, fruit skin coloring period, harvesting period and acidity. However, the fruits of aforesaid ‘Jedae mandarin’ has v) radial grooves in the fruit stalk and fruit skin with a longitudinally raised part resulting in formation of fruit skin with vertical corrugation. vi) The raised part of aforesaid fruit skin is characterized by an anatomical and morphological development of vascular bundles.

Additionally, the fruit of ‘Jedae mandarin’ is characterized by vii) smoother surface of the area of albedo layer which comes into contact with fruit flesh, compared to that of C. unshiu Marc. cv. Miyagawa-wase, viii) greater fruit skin hardness in comparison to the hardness of C. unshiu Marc. cv. Miyagawa-wase, and ix) tendency of cytolysis of epidermal cells in mature fruit, compared to C. unshiu Marc. cv. Miyagawa-wase.

Furthermore, the fruit of ‘Jedae mandarin’ is characterized by: x) good adhesion between fruit flesh and fruit skin, xi) low incidence of fruit skin diseases, and xii) good fruit storeability. The fruit of aforesaid ‘Jedae mandarin’ is also distinguished by: xiii) similar or slightly larger fruit size, compared to C. unshiu Marc. cv. Miyagawa-wase and xiv) similar or slightly greater thickness of fruit skin, compared to C. unshiu Marc. cv. Miyagawa-wase.

Fruit of ‘Jedae mandarin’ has general characteristics as below: 1) Longitudinal diameter: 35 to 70 mm, 2) Transverse diameter: 45 to 80 mm, 3) Weight: 70 to 150 g, 4) Fruit skin thickness: 1.5 to 3.5 mm, 5) Sugar content: 8 to 13° Brix, 6) Acidity: 0.7 to 1.1 wt %, 7) Propagation method: asexual propagation through grafting.

Citrus tree of ‘Jedae mandarin’ has following characteristics: 8) Blooming period: early May to mid May, 9) Fruit skin coloration period: mid November to late November, 10) Harvest period: late November to mid December.

Overall shape, leaf, and petiole of ‘Jedae mandarin’ citrus tree are characterized by: 11) Overall shape: Tree growth habit is in open-grown stand with very little or sparse thorn density, 12) Length of leaf: 97.5±7.0 mm on average, 13) Width of leaf: 40.3±4.8 mm on average, 14) Shape of leaf: Leaves have oval shape with slightly concave cross-section of leaf blade. The intensity of greenness is moderate, and apical part of leaf blade has a pointed shape, 15) Length of petiole: 18.6±1.5 mm on average. It has a morphological characteristic that the pinnate is absent(see FIG. 17). However, such characteristics may vary slightly due to the influence of weather or environment, but the scope of appended claims is not limited by these differences.

Considering the characteristics of fruit shape and fruit quantity described above, the ‘Jedae mandarin’ of the present invention can be cultivated for reproduction purpose due to its quality similar that of C. unshiu Marc. cv. Miyagawa-wase, currently being farmed widely, in terms of sugar content and acidity, and additionally, has a unique fruit shape that can be developed for decoration purpose. Moreover, ‘Jedae mandarin’ of the present invention has high content of flavonoid, a functional ingredient, and therefore can be used as a functional food or a processing material necessary for functionalities of foods.

Aforesaid Jedae mandarin, a new variety of early-ripening satsuma mandarin, has the fruit traits similar to those of C. unshiu Marc. cv. Miyagawa-wase, which results in the fruit coloring period spanning from late November to early December. However, such characteristics of Jedae mandarin may vary, depending on climate, soil, environment, cultivation method, fruit size and age of tree.

Aforesaid Jedae mandarin can be bred by radiation irradiation of branches or buds of C. unshiu Marc. cv. Miyagawa-wase, grafting the irradiated buds to adult tree to induce branching, collecting the buds from the branches that have mutant fruits with altered shapes, and grafting the collected buds of those branches to rootstocks of trifoliate orange tree, or can be asexually propagated stably by grafting the buds of branches from mutant plant, bred as described above, onto the rootstocks.

The branch buds of aforesaid C. unshiu Marc. cv. Miyagawa-wase may be irradiated with ⁶⁰Co gamma radiation at a dose of 40 Gray(0.4 rad), and above-mentioned grafting can be performed by using various citrus trees. Specifically, aforesaid Stage 1) can be carried out by irradiating ⁶⁰Co gamma rays(y-ray) to the buds of branches of C. unshiu Marc. cv. Miyagawa-wase with a dose of 40Gy at a distance of 0.68 m from a radiation source for 22 hours.

The flavonoid, a functional substance contained in citrus fruits, is typified by components such as hesperidin(HD) and narirutin(NRT). Contained in fruit skin or fruit flesh of citrus, flavonoid has benevolent effect, such as anti-oxidation and hyperlipidemia inhibition effect, on human body.

The fruit of aforesaid Jedae mandarin has higher content of flavonoid, compared to that contained in the fruit of C. unshiu Marc. cv. Miyagawa-wase cultivated under the same conditions. In addition, the contents of hesperidin(HD) and narirutin(NRT) are contained more in the fruit of aforesaid Jedae mandarin than in the fruit of C. unshiu Marc. cv. Miyagawa-wase cultivated under the same conditions.

Mature fruits of the variant of citrus plant named ‘Jedae mandarin’ may exhibit different characteristics, depending on environmental conditions such as weather and soil, or age of tree. However, those fruits can achieve the traits including the fruit weight of 70-150 g, fruit diameter of 35-80 mm(longitudinal diameter of fruit: 35-70 mm; transverse diameter of fruit: 45-80 mm), fruit skin thickness of 1.5-3.5 mm, sugar content of 8-13° Brix, and acidity of 0.7-1.1 wt %, and can be asexually propagated with grafting method.

The citrus in accordance with another embodiment of the present invention is harvested from a variant of citrus plant named ‘Jedae mandarin’ as described above. More detailed description of aforesaid citrus is omitted because it is redundant with the characteristics of fruit of Jedae mandarin already described above.

The method for breeding a variant of citrus plant named ‘Jedae mandarin’ in accordance with another embodiment of the present invention includes the following stages: 1) a stage in which buds of branches in C. unshiu Marc. cv. Miyagawa-wase are irradiated with gamma rays; 2) a stage in which buds, irradiated with gamma rays as described in the foregoing stage 1), are grafted to induce branching and thereafter the buds are collected from mutant branches that have fruits with altered shape; and 3) a stage in which the mutant branches' buds, selected as described in the foregoing stage 2), are grafted onto the rootstocks. In that way, the variant of citrus plant named ‘Jedae mandarin’ can be bred which has the characteristics described above. More detailed description of aforesaid citrus is also omitted because it is redundant with the fruit of Jedae mandarin already described above.

The fruit of mutant citrus named ‘Jedae mandarin’ is an early-maturing satsuma mandarin characterized by i) rough peel, ii) vertical corrugation from fruit stalk which results in uneven outer appearance of fruit, and iii) higher flavonoid content in the fruit, compared to flavonoid content in the fruit of C. unshiu Marc. cv. Miyagawa-wase. More detailed description of aforesaid citrus is omitted because it is redundant with the characteristics of fruit of Jedae mandarin already described above.

The branch buds of aforesaid C. unshiu Marc. cv. Miyagawa-wase may be irradiated with ⁶⁰Co gamma radiation at a dose of 40 Gray(0.4 rad), and above-mentioned grafting can be performed by using various citrus trees such as adult trees, trifoliate orange trees or swingle, etc. For instance, the grafting described in the stage 1) can be performed by using adult trees while the grafting described in stage 2) can be carried out by using the trifoliate orange trees.

the variant of citrus plant named ‘Jedae mandarin’ in accordance with another embodiment of the present invention can be asexually reproduced and propagated through the processes including the process of grafting the branch buds of a variant of citrus plant named ‘Jedae mandarin’ which have characteristics described above.

At this time, aforesaid rootstocks can be utilized to ensure stable asexual reproduction and propagation of named ‘Jedae mandarin’ although the buds mentioned above can be grafted onto the rootstocks of various citrus trees such as adult trees or swingle.

The fruit of mutant citrus named ‘Jedae mandarin’ is an early-maturing satsuma mandarin characterized by i) rough peel, ii) vertical corrugation from fruit stalk which results in uneven outer appearance of fruit, and iii) higher flavonoid content in the fruit, compared to flavonoid content in the fruit of C. unshiu Marc. cv. Miyagawa-wase. More detailed description of aforesaid citrus is omitted because it is redundant with the characteristics of fruit of Jedae mandarin already described above.

Aforesaid Jedae mandarin is similar to early-ripening satsuma mandarin in terms of blooming period, shape of leaves, type of flowers, cultivation environment, etc., but has characteristics different from those of C. unshiu Marc. cv. Miyagawa-wase in terms of fruit shape and flavonoid content. In other words, the fruit of Jedae mandarin exhibits the property of uneven surface of fruit skin and thicker fruit skin and has greater contents of hesperidin and narirutin which have been reported to have functionalities such as anti-cancer effect among flavonoid contents.

Thus, Jedae mandarin inherits genetic traits of mother plant and therefore can be mass-produced stably, and furthermore, can be asexually propagated by grafting. In addition, Jedae mandarin is a new that can be used for decoration purpose due to the changes in its fruit shape, as well as reproduction purpose, and can be also used as a material for functional food processing, depending on variation of flavonoid content.

### Advantageous Effects

The mutant citrus named ‘Jedae mandarin’, an early-maturing satsuma mandarin of the present invention, has fruits with shape different from that of ordinary C. unshiu Marc. cv. Miyagawa-wase currently being cultivated widely, and is a new variety with altered flavonoid content and has excellent economic value.

### DESCRIPTION OF DRAWINGS

FIG. 1 is a photograph showing the breeding process of named ‘Jedae mandarin’ bred in accordance with the Embodiment 1 of the present invention.

FIG. 2 is a photograph taken on Jul. 2, 2013, showing the fruit bearing of named ‘Jedae mandarin’ bred in accordance with Embodiment 1 of the present invention (cultivated in Donghong-dong, Seogwipo-si, Jeju Special Self-Governing Province, Korea).

FIG. 3 is a photograph taken on Nov. 20, 2013, showing the fruit bearing of named ‘Jedae mandarin’ bred in accordance with Embodiment 1 of the present invention (cultivated in Donghong-dong, Seogwipo-si, Jeju Special Self-Governing Province, Korea).

FIG. 4 is a photograph taken in early July 2013, showing the fruit of mutant citrus named ‘Jedae mandarin’ bred in accordance with Embodiment 1 of the present invention.

FIG. 5 is a photograph taken in early November 2013, showing the fruit of mutant citrus named ‘Jedae mandarin’ bred in accordance with Embodiment 1 of the present invention.

FIG. 6 is a photograph taken in early December 2013, showing the fruit of mutant citrus named ‘Jedae mandarin’ bred in accordance with Embodiment 1 of the present invention.

FIG. 7 is a photograph taken in early December 2013, showing the fruit of C. unshiu Marc. cv. Miyagawa-wase cultivated under the conditions same as those for Jedae mandarin bred in accordance with Embodiment 1 of the present invention.

FIG. 8 is a side view illustration of mature fruit of mutant citrus named ‘Jedae mandarin’ bred in accordance with Embodiment 1 of the present invention.

FIG. 9 is a photograph comparing the difference in external appearance of stalk(top) of citrus between the fruit of mutant citrus named ‘Jedae mandarin’ bred in accordance with Embodiment 1 of the present invention(on the right side) and the fruit of C. unshiu Marc. cv. Miyagawa-wase(on the left side).

FIG. 10 is a photograph comparing the difference in external appearance of the rear(bottom) of citrus between the fruit of mutant citrus named ‘Jedae mandarin’ bred in accordance with Embodiment 1 of the present invention(on the right side) and the fruit of C. unshiu Marc. cv. Miyagawa-wase(on the left side).

FIG. 11 is a photograph comparing the difference in citrus fruit cross-section of between mutant citrus named ‘Jedae mandarin’ bred in accordance with Embodiment 1 of the present invention and C. unshiu Marc. cv. Miyagawa-wase(top and left side: C. unshiu Marc. cv. Miyagawa-wase/right side: Jedae mandarin).

FIG. 12 is a photograph comparing the difference in white area(albedo layer) inside fruit peel between mutant citrus named ‘Jedae mandarin’ bred in accordance with Embodiment 1 of the present invention and C. unshiu Marc. cv. Miyagawa-wase(bottom and left side: C. unshiu Marc. cv. Miyagawa-wase/right side: Jedae mandarin).

FIG. 13 is a photograph(20×) showing the results of microscopic observation of the peel of young fruit(harvested in June 2013) of C. unshiu Marc. cv. Miyagawa-wase at cellular level in accordance with Embodiment 3 of the present invention.

FIG. 14 is a photograph(20×) showing the results of microscopic observation of the peel of young fruit(harvested in June 2013) of Jedae mandarin at cellular level in accordance with Embodiment 3 of the present invention.

FIG. 15 is a photograph(40×) showing the results of microscopic observation of the peel of mature fruit(harvested in December 2013) of C. unshiu Marc. cv. Miyagawa-wase at cellular level in accordance with Embodiment 3 of the present invention(the arrow indicates the epidermal cell cytolysis).

FIG. 16 is a photograph(40×) showing the results of microscopic observation of the peel of mature fruit(harvested in December 2013) of Jedae mandarin at cellular level in accordance with Embodiment 3 of the present invention.

FIG. 17 is a photograph comparing the difference in leaf shape between Jedae mandarin citrus tree bred in accordance with this Embodiment of the present invention and C. unshiu Marc. cv. Miyagawa-wase citrus tree.

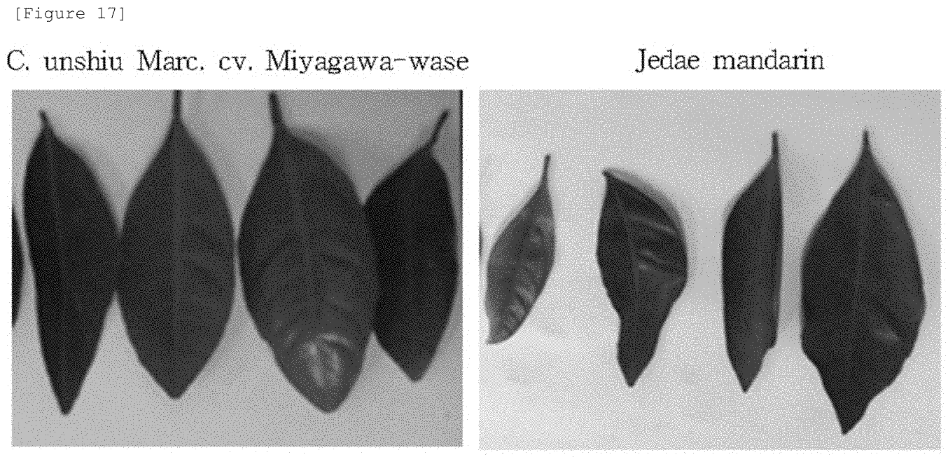

### BEST MODE

Hereinafter, exemplary embodiments of the present disclosure were described in detail by referring to the accompanying drawings to increase the ease of embodiment by those with general knowledge in concerned technical fields of the present invention. The present invention may, however, be embodied in many different forms and is not limited to the embodiments set forth herein.

### <Embodiment 1> Breeding of Citrus Mutated by Radiation

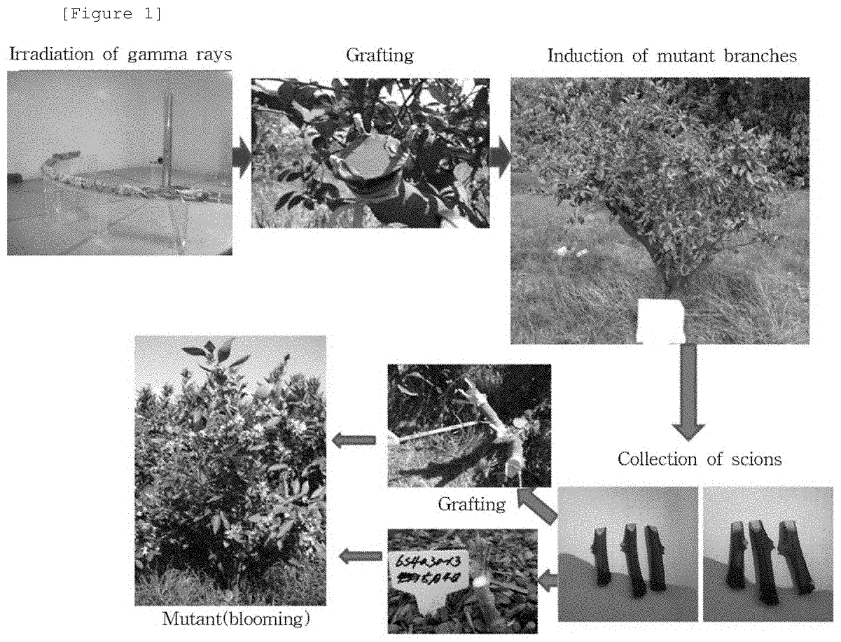

FIG. 1 is a photograph showing the breeding process of named ‘Jedae mandarin’ bred in accordance with the Embodiment 1 of the present invention. The mutant citrus breeding process using radiation was described by referring to FIG. 1 hereinafter.

In the present invention, C. unshiu Marc. cv. Miyagawa-wase, cultivated the most widely in Jeju Province, was selected as a material for a breeding experiment. Thus, 5 kg of branches of C. unshiu Marc. cv. Miyagawa-wase of early-ripening satsuma mandarin was obtained from the Agricultural Research & Extension Services of Jeju Special Self-Governing Province for use in aforesaid experiment. At this time, the branches of C. unshiu Marc. cv. Miyagawa-wase used in the experiment had the scions collected for the period between early February and mid February for radiation-induced mutation of buds. The branches, collected in that way, were stored in 4 refrigerated conditions for 3 months from mid April to early May, the period of grafting, before being used.

The buds of branches of C. unshiu Marc. cv. Miyagawa-wase, obtained from the Agricultural Research & Extension Services of Jeju Special Self-Governing Province, were irradiated with gamma irradiation at the cobalt-60(⁶⁰Co) irradiation facility(source: C-188, Nordion International Ltd., Canada) in the Radiation & Applied Science Laboratory(currently, the Institute for Nuclear Science & Technology) of Jeju National University to induce mutation. Then, the irradiated buds mentioned above were joined with adult trees of C. unshiu Marc. cv. Miyagawa-wase through top-grafting. Cultivation and maintenance were carried out in accordance with customary methods.

Specifically, mutation was induced in the buds of branches of C. unshiu Marc. cv. Miyagawa-wase with gamma irradiation from the Cobalt-60(⁶⁰Co) irradiation facility at Radiation & Applied Science Laboratory of Jeju National University in April 2006. At this time, the buds of branches of C. unshiu Marc. cv. Miyagawa-wase were irradiated with gamma rays emitted at a distance of 0.68 m from the radiation source at a dose of 40 Gy for 22 hours. The buds of branches of C. unshiu Marc. cv. Miyagawa-wase, which were irradiated with gamma rays to induce mutation, were grated onto adult trees of C. unshiu Marc. cv. Miyagawa-wase which had been used as rootstock at Dongheung-dong packaging room of the Research & Practice Center affiliated with Jeju National University to induce branching. Selection of branches was performed after investigation of their traits. A total of 889 scions were grafted. Total germination rate stood at 80.3%(714 buds in all) as found in the germination rate survey after 2 months from the date of grafting.

The buds irradiated with gamma ray were grafted onto adult trees of C. unshiu Marc. cv. Miyagawa-wase(citrus packaging room of the Research & Practice Center affiliated with Jeju National University) to induce branching, and then the mutant branches were selected upon investigation of the traits based on whether the fruit shape was changed.

In addition, fruits generated from each mutant branch were stored in a low-temperature storage(5° C.) after they were harvested from late November to early December every year during the period spanning between 2009 and 2014.

Moreover, weight, diameter, peel thickness, sugar content, acidity, color, etc., of the fruits were measured.

The weight of fruit was measured up to 0.1 g unit by using an electronic indicator scale(CAS Co., Ltd, Korea). The longitudinal diameter and transverse diameter were measured up to 0.1 mm by using the digital caliper(Mitutoyo Corporation, Japan).

After measuring the diameter and thickness of each fruit, juice was collected from each fruit. Sugar content and acidity were measured by the acidity and sugar content analyzer NH-2000(HORIBA, Japan), and 4-5 mL of juice was inserted into the syringe as specified in the operation manual of the device.

Additionally, chroma meter(CR-400, Minolta) was used to measure the chromaticity of mutant fruits that exhibited changes in fruit color.

Changes in fruit shape were observed through naked eyes, while changes in the fruit peels were observed additionally at the cellular level with a microscope(Nikon microphoto type 114).

Mutant buds were collected again from the branches selected as mentioned above, and then, were grafted again onto rootstocks of adult trees and trifoliate orange trees(purchased from seedling suppliers) to induce fruit-bearing of citrus(see FIG. 1).

### <Embodiment 2> Analyses on Characteristics of Mutant Citrus, an Early-Maturing Satsuma Mandarin

1) Comparison of General Traits

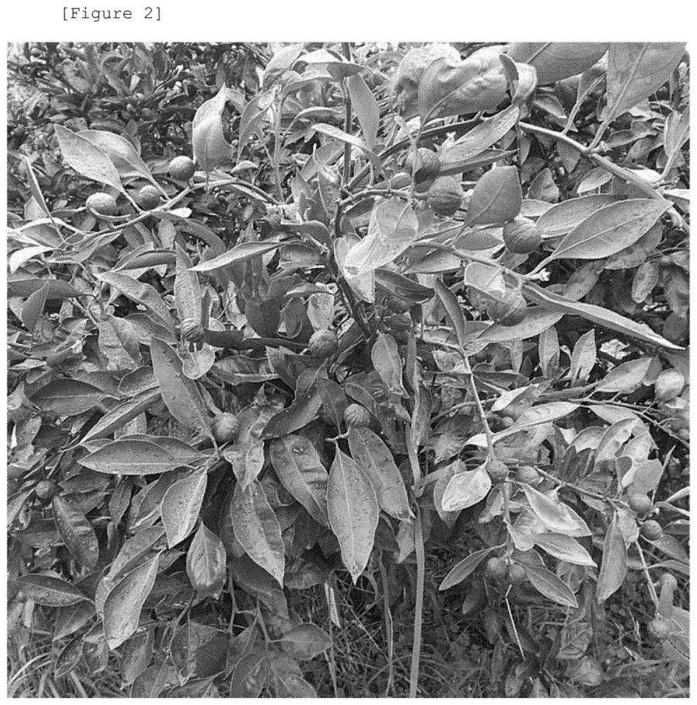

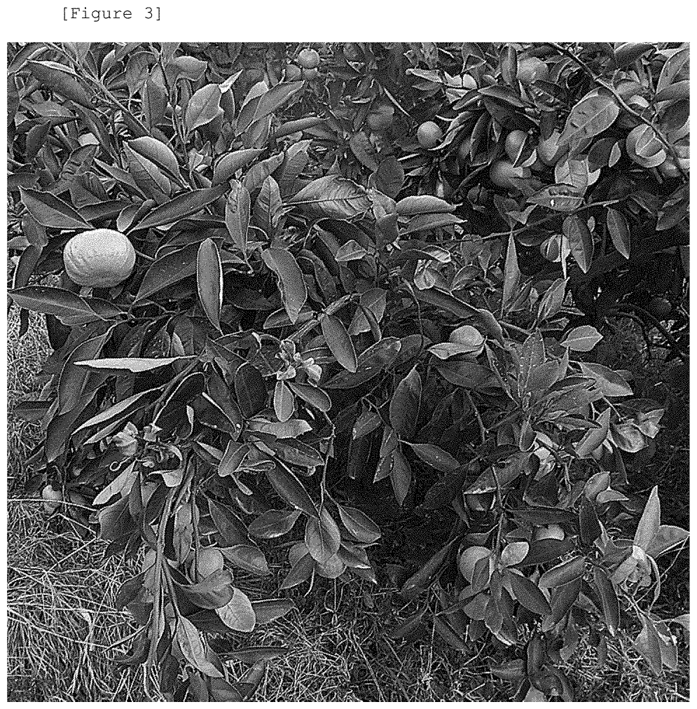

The citrus fruits produced in accordance with aforesaid Embodiment 1 were characterized by comparing general traits of citruses and fruit trees, which bore fruits in 2013, with those of C. unshiu Marc. cv. Miyagawa-wase(see FIG. 2 and FIG. 3).

Specifically, the mutant citrus bred in accordance with aforesaid Embodiment 1 showed characteristics similar to those of C. unshiu Marc. cv. Miyagawa-wase, cultivated commonly in Jeju Province, in terms of general traits such as leaf shape, leaf color, blooming period, vitality of tree, etc., as shown in Table 1 below.

TABLE 1

| | Vitality | Blooming | Coloring | Period of |
|---|---|---|---|---|
| Variety | of Tree | Period | of Peel | Harvesting |
| C. unshiu | Moderate | From May | Late | From late |
| Marc. cv. | | 10 to 20 | November | November to |
| Miyagawa-wase | | | | mid December |
| Jedae mandarin | Moderate | From May | Late | From late |
| | | 10 to 20 | November | November to |
| | | | | mid December |

2) Comparison of Characteristics of Fruits

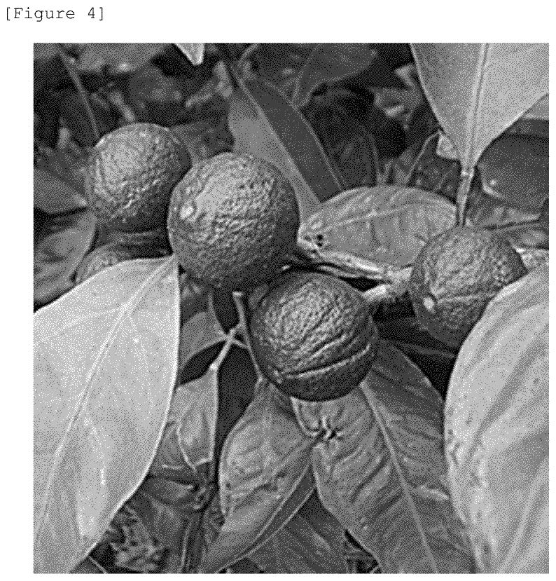

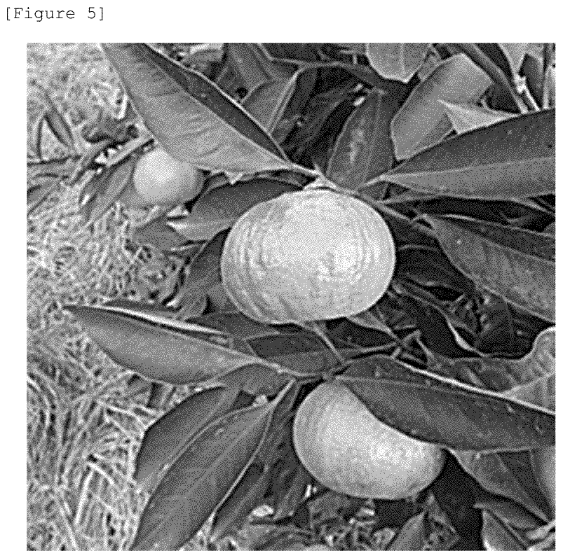

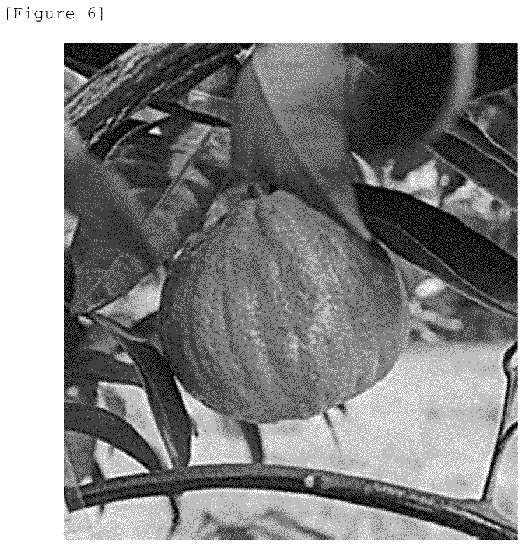

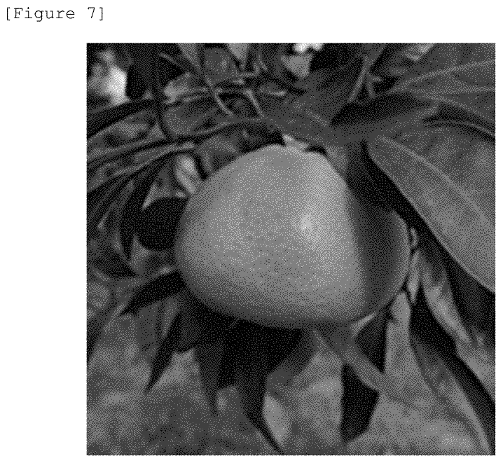

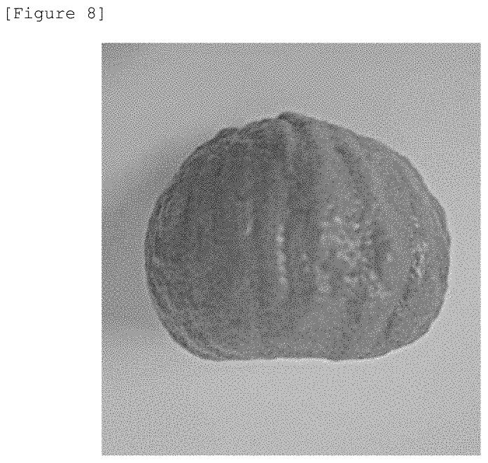

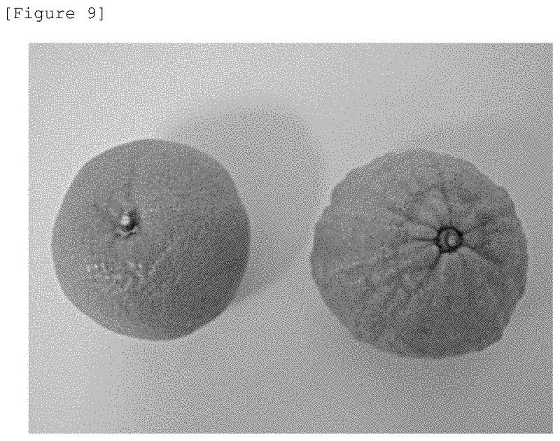

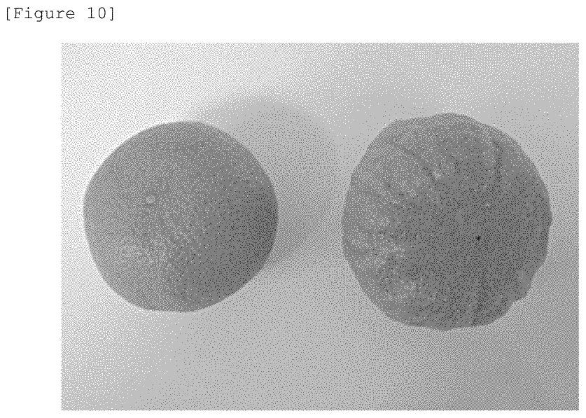

FIG. 4 to FIG. 7 present the photographs of fruits of mutant citrus named ‘Jedae mandarin’ bred in accordance with the Embodiment 1 of the present invention, which were taken in early July, early November, and early December, respectively, and the photographs of fruits of C. unshiu Marc. cv. Miyagawa-wase, the mother plant, which were taken in early December. Moreover, FIG. 8 shows side view photograph of mature fruit of Jedae mandarin and FIG. 9 shows the outer appearance of citrus leaf stalk which is compared with that of C. unshiu Marc. cv. Miyagawa-wase. FIG. 10 shows the photograph that compares the outer appearance of rear part of the citrus with that of C. unshiu Marc. cv. Miyagawa-wase.

Besides, the mutant citruses, which had been bred in accordance with aforesaid Embodiment 1 of the present invention and thereafter bloomed between May 10 and May 20(cultivated on open field in Donghong-dong, Seogwipo-si, Jeju Special Self-Governing Province), were harvested in early December to mid December for the period of 4 years. Harvested mutant citruses were investigated to determine their traits, and the results were summarized in Table 2.

TABLE 2

| | Longitudinal | Transverse | | | | | | | | |
|---|---|---|---|---|---|---|---|---|---|---|
| | Diameter of | Diameter of | Fruit | Peel | Sugar | | Hunter Color Value | | | |
| | Fruit | Fruit | Weight | Thickness | Content | Acidity | L | a | b | Harvesting |
| Year | (mm) | (mm) | (g) | (mm) | (°Brix) | (wt %) | (Lightness) | (Redness) | (Yellowness) | Period |

Jedae mandarin

| | | | | | | | | | | |
|---|---|---|---|---|---|---|---|---|---|---|
| 2009 | 62.3 | 78.1 | 156.73 | 4.2 | 10.2 | 0.97 | 64.41 | 33.95 | 64.66 | Mid |
| | | | | | | | | | | December |
| 2010 | 54.8 | 66.5 | 131.6 | 2.1 | 7.8 | 0.78 | 67.78 | 29.56 | 72.44 | Early |
| | | | | | | | | | | December |
| 2011 | 52.8 | 62.6 | 110.3 | 2.6 | 10.8 | 0.69 | 69.5 | 26.5 | 72.4 | Early |
| | | | | | | | | | | December |
| 2013 | 34.9 | 46.3 | 41.1 | 2.5 | 11.8 | 0.85 | 66.48 | 30.69 | 68.77 | Early |
| | | | | | | | | | | December |
| 2014 | 65.35 | 64.64 | 126.3 | 4.02 | 7.4 | 1.25 | 59.55 | 30.51 | 34.84 | Early |
| | | | | | | | | | | December |

C. unshiu Marc. cv. Miyagawa-wase

| | | | | | | | | | | |
|---|---|---|---|---|---|---|---|---|---|---|
| 2009 | 43.7 | 50.1 | 67.4 | 2.3 | 8.2 | 0.83 | 66.88 | 26.96 | 73.37 | Early |
| | | | | | | | | | | December |
| 2010 | 51.7 | 56.6 | 94.0 | 2.0 | 8.8 | 1.00 | 67.56 | 31.13 | 70.31 | Early |
| | | | | | | | | | | December |
| 2011 | 50.1 | 60.0 | 94.6 | 2.4 | 9.6 | 0.9 | 39.36 | 28.77 | 72.18 | Early |
| | | | | | | | | | | December |
| 2013 | 45.5 | 60.4 | 88.2 | 2.4 | 10.2 | 0.82 | 67.58 | 32.36 | 69.57 | Early |
| | | | | | | | | | | December |
| 2014 | 57.6 | 61.38 | 102 | 3.44 | 7.4 | 1.25 | 58.33 | 30.74 | 33.32 | Early |
| | | | | | | | | | | December |

FIG. 4 to FIG. 10, and Table 2 above showed that fruits of Jedae mandarin bred in accordance with the Embodiment 1 had the shape different from that of C. unshiu Marc. cv. Miyagawa-wase, the mother plant. In particular, the photographs of Jedae mandarin fruits in the drawings indicated that the fruit shape of Jedae mandarin was different significantly from that of C. unshiu Marc. cv. Miyagawa-wase, the mother plant, from which the mutation had occurred, depending on variations of peel traits.

Specifically, fruits of Jedae mandarin exhibited characteristics almost similar to those of fruits of C. unshiu Marc. cv. Miyagawa-wase in terms of sugar content and acidity. Fruits of Jedae mandarin were slightly larger than those of C. unshiu Marc. cv. Miyagawa-wase. In addition, fruits of Jedae mandarin had the peels that were less smooth and more rugged on outer surface in terms of fruit shape, compared to fruits of C. unshiu Marc. cv. Miyagawa-wase.

Fruits of Jedae mandarin had groove of radial shape from the fruit stalk and were found to have vertical corrugation in overall way when they were viewed from the side. Thus, fruits of Jedae mandarin were considered to have excellent fruit shape and have the potential to be developed into decorative citrus fruits.

In addition, fruits generated from Jedae mandarin maintain their altered shapes described above from young fruit bearing stage to maturation stage in early December, and can be harvested with those of C. unshiu Marc. cv. Miyagawa-wase almost around the same time. However, fruits of Jedae mandarin showed distinctive difference from those of C. unshiu Marc. cv. Miyagawa-wase in terms of fruit shape arising from changes in peel traits.

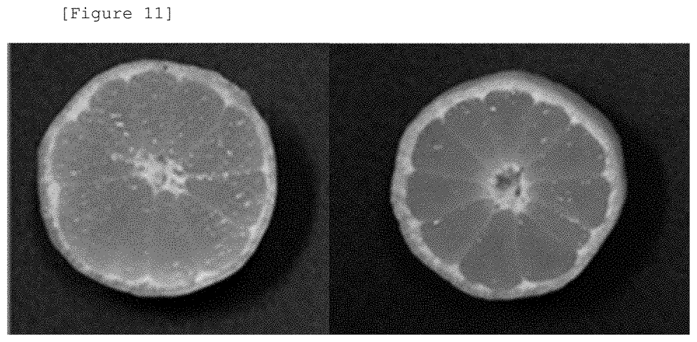

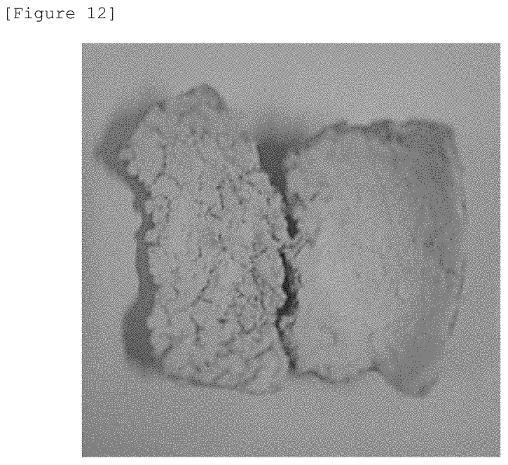

FIG. 11 shows the photograph that compares the citrus section of mutant citrus named ‘Jedae mandarin’ bred in accordance with the Embodiment 1 of the present invention with the fruit section of C. unshiu Marc. cv. Miyagawa-wase(top and left side: C. unshiu Marc. cv. Miyagawa-wase; right side: Jedae mandarin). FIG. 12 shows the photograph comparing the white portion(albedo layer) inside the peel with that of C. unshiu Marc. cv. Miyagawa-wase(bottom and left side: C. unshiu Marc. cv. Miyagawa-wase; right side: Jedae mandarin).

FIG. 11 and FIG. 12 above show that the mutant citrus named ‘Jedae mandarin’ is also characterized by smooth fiber not protruding roughly, regarding the surface that comes into contact with fruit flesh in albedo layer, unlike C. unshiu Marc. cv. Miyagawa-wase. Therefore, fruits of Jedae mandarin showed good adhesion between fruit and fruit flesh and good storability, but could not be easily peeled.

As shown in Table 2 and FIG. 4 through FIG. 12, the fruits of Jedae mandarin, a mutant citrus bred in accordance with aforesaid Embodiment 1, exhibited difference in traits, depending on climate (amount of precipitation, amount of sunshine, etc.) and fruit size of the year where irradiation occurred. However, the changes in fruit shape were found to be maintained stably.

3) Comparison of Content of Functional Component(Flavonoid)

The harvested citrus fruits were washed twice with water to remove pesticides and other impurities. Fruit flesh and peel were separated and sliced to the thickness of 0.3 cm and then frozen for 3 days in a −70° C. ultra-low temperature refrigerator, followed by lyophilization(freeze-drying) for 72 hours. 100% methanol was selected as the extraction solvent based on the studies, such as the study by Senevirathne(2010, Journal of Food Engineering, 97, 168-176), et al., which involved the use of citrus, among existing literatures on analyses of flavonoid.

For extraction, the lyophilized portion of citrus(peel and fruit flesh) were pulverized with a pulverizer and powdered by using 0.5 mm mesh. 250 mL of 100% methanol was added to 2.5 g of the powdered citrus samples(10 mg/mL each). Then, flavonoid was extracted using a shaking incubator at 25° C. for 24 hours and the solid contents were removed. Then, the flavonoid content was used for analyses with HPLC system mentioned below.

The HPLC system used a 600 pump from Waters(Milford, Mass.). Flavonoid was detected at a wavelength of 280 nm using the W2998 UV-VIS detector.

Shim-pack VP-ODS(C18) column(4.6×150 mm, 5 μm) of Shimadzu Corporation was used for analyses. Chromatograms were prepared with HPLC by using the 7 standard substances(rutin, naringin, neohesperidin, hesperidin, narirutin, naringenin, and hesperetin) purchased from Sigma(St. Louis, Mo., US), and then the best detection wavelengths were selected. That was followed by establishment of mobile phase conditions[Eluent A, acetonitrile/acetic acid(1000/5); Eluent B, DW/acetic acid(1000/5); flow rate, 1 mL/min] suitable for analyses of flavonoid for isolation of peaks. Based on the established mobile phase conditions, 10 μL of each extract was injected, followed by analyses using the standard curve.

Table 3 shows respective contents of hesperidin(HD) and narirutin(NRT) in citrus peel, which were extracted as described above. Table 4 shows respective contents of hesperidin(HD) and narirutin(NRT) in citrus fruit flesh. All experiments were repeated 3 times. Experimental values were expressed as mean value and standard deviation value. ANOVA(Analysis of Variance) was performed using the SPSS program(12.0), and concerned significant values were verified by Duncan's multiple range test(p<0.05).

TABLE 3
Content

| (mg/100 g dry | 2010 | | 2014 | |
|---|---|---|---|---|
| weight of peel) | HD | NRT | HD | NRT |
| Control | 463.1 ± 1.11 | 60.8 ± 1.51 | 305.3 ± 0.85 | 22.2 ± 1.34 |
| (C. unshiu | | | | |
| Marc. cv. | | | | |
| Miyagawa-wase) | | | | |
| Jedae mandarin | 546.6 ± 3.38* | 78.5 ± 0.61* | 431.6 ± 2.37* | 43.7 ± 0.82* |

*shows the value with significant difference in comparison to the control (p < 0.05).

TABLE 4

| Content | | | | |
|---|---|---|---|---|
| (mg/100 g dry | 2010 | | 2014 | |
| weight of fruit flesh) | HD | NRT | HD | NRT |
| Control | 178.8 ± 0.43 | 92.4 ± 0.59 | 75.3 ± 0.85 | 12.2 ± 1.34 |
| (C. unshiu | | | | |
| Marc. cv. | | | | |
| Miyagawa-wase) | | | | |
| Jedae mandarin | 276.3 ± 2.07* | 132.2 ± 1.53* | 125.6 ± 1.01* | 22.4 ± 0.29* |

*shows the value with significant difference in comparison to the control (p < 0.05).

As shown in Tables 3 and Table 4, the contents of hesperidin and narirutin among flavonoid components varied, depending on the harvest year in which the amount of precipitation and sunshine differed. However, their contents in mutant citrus which had been bred were significantly higher than those in C. unshiu Marc. cv. Miyagawa-wase which was the control.

### <Embodiment 3> Cell-Level Microscopic Characterization of the Peel of Mutant Citrus, an Early-Maturing Satsuma Mandarin

Observation of Delayed Decay of Fruit Peel

The observation materials were the young fruits and maturated fruits of Jedae mandarin and C. unshiu Marc. cv. Miyagawa-wase harvested altogether in 2013. Outer peel of citrus fruit was cut into the size of 5 mm×5 mm and fixed in FAA solution. Then, the cross-section of 10 μm in thickness was prepared by Paraffin method, followed by double staining with Safranine and Fast Green and microscopic examination.

Fixation was carried out by applying the FAA method. Each plant slice was cut into the size of 5 mm×5 mm and placed in a FAA fixative solution containing 90 ml of 50% alcohol, 5 ml of formalin, and 5 ml of acetic acid, and then, were fixed for 24-72 hours through vacuum processing which aimed to remove the air from the inside of plant tissues.

The fixed tissue was treated for 5 hours in 50% alcohol and then dehydrated for the following hours: for 5 hour in solution I (10 ml of tert-butyl alcohol, 40 ml of 95% alcohol, 50 ml of distilled water); for 37 hours in solution II (20 ml of tert-butyl alcohol, 50 ml of 95% alcohol, 30 ml of distilled water); for 5 hours in solution III (35 ml of tert-butyl alcohol, 50 ml of 95% alcohol, 25 ml of distilled water); for 18 hours in solution IV (10 ml of tert-butyl alcohol, 45 ml of 55% alcohol, crystol violet 1%); for 5 hours in solution V (75 ml of tert-butyl alcohol, 25 ml of 100% alcohol); and for 5 hours and 72 hours in solution VI (100 ml of tert-butyl alcohol).

The paraffin solution, which had already been dissolved in the dehydrated tissue, was slowly poured, allowing the paraffin to slowly penetrate into the tissue. For the tissue sectioning, the transverse slices, each with a cross-section of 10 μm in thickness, were prepared by using the microtome.

Double staining with Safranine and Fast Green was carried out twice for 60 minutes and 10 minutes, respectively, in xylene. The slices were treated for 10 minutes in Xylene:Alcohol and for 10 minutes, respectively, in 100%, 95%, 85%, and 75% alcohol. Then, they were treated with 1% Safranine for 2 hours, 75% alcohol for 5 minutes, 0.5% Fast Green for 30 seconds, 95%, 100% and 100% alcohol for 5 minutes each.

In addition, they were treated twice, i.e., with Xylene:Alcohol for 5 minutes and then with Xylene for 10 minutes. Following that, they were observed with a microscope(Nikon microphoto type 114).

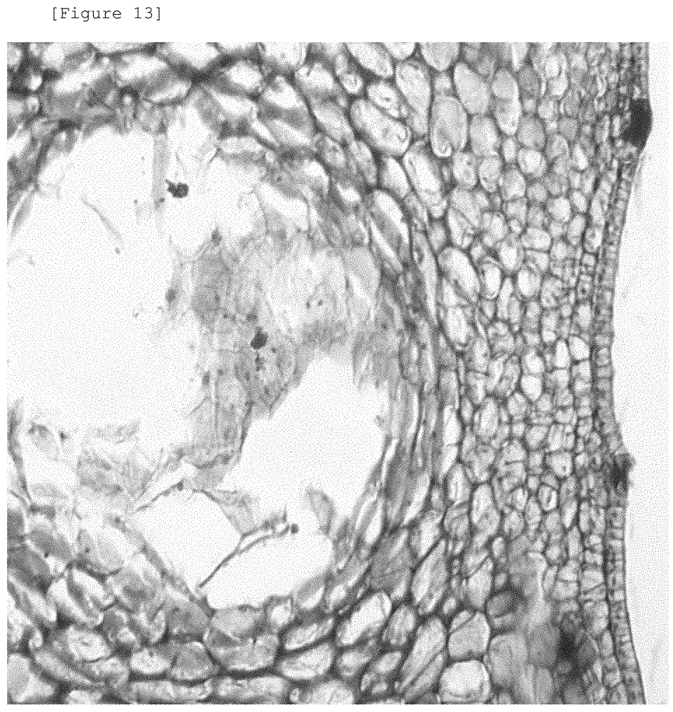

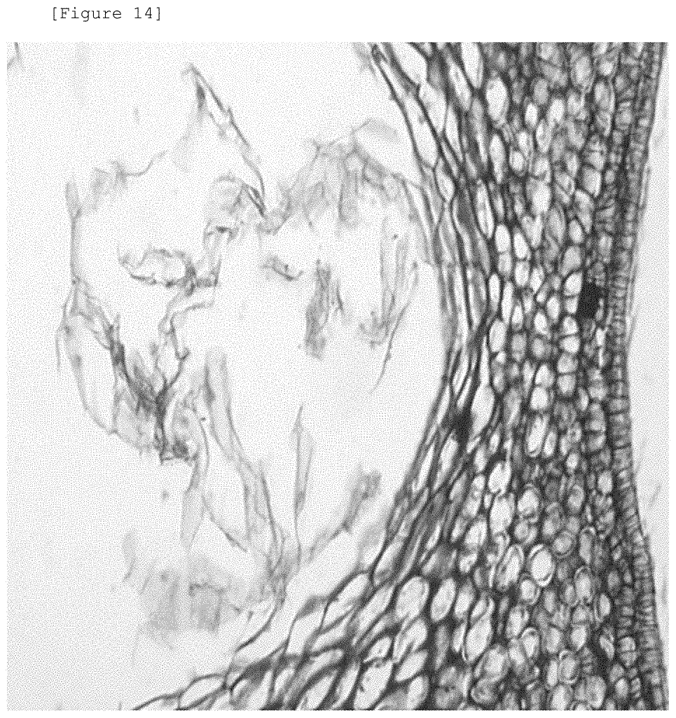

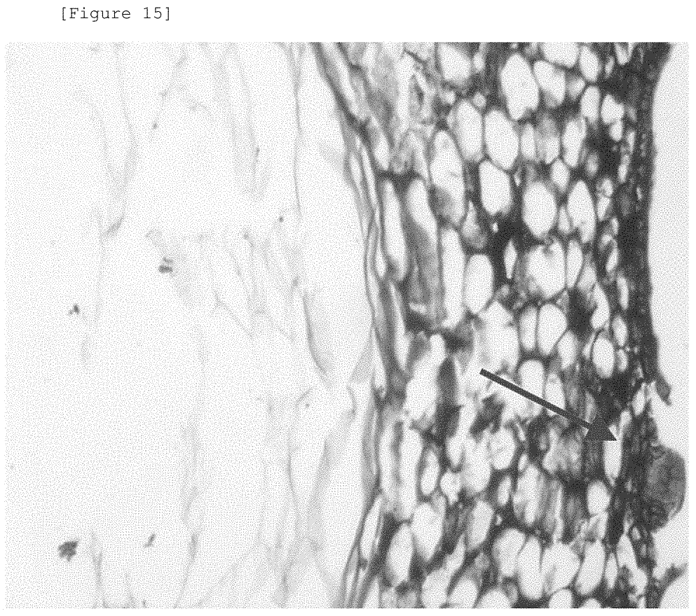

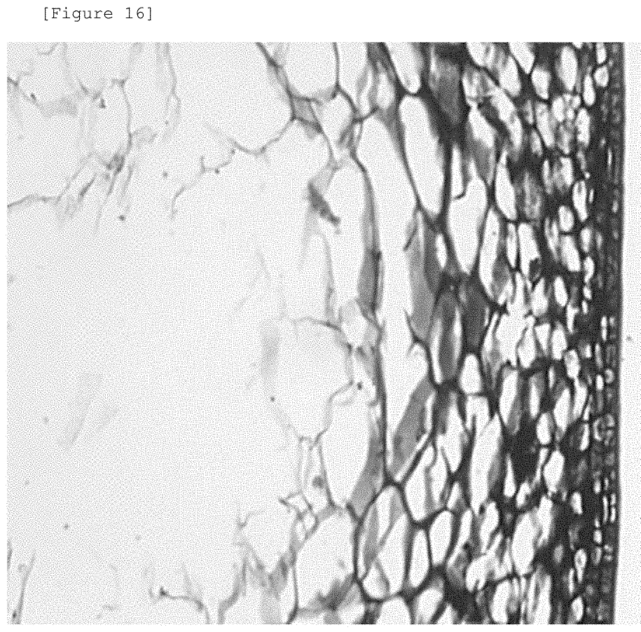

By using the method described above, cell-level microscopic observation was performed of the peel shape of young fruits of C. unshiu Marc. cv. Miyagawa-wase(harvested in June 2013), young fruits of Jedae mandarin(harvested in June 2013), mature fruits of C. unshiu Marc. cv. Miyagawa-wase(harvested in December, 2013), and mature fruits of Jedae mandarin(harvested in December, 2013). The results are presented in FIG. 13 through FIG. 16.

When the peels of C. unshiu Marc. cv. Miyagawa-wase(control group) and those of Jedae mandarin(experiment group) were compared by referring to the FIG. 13 through FIG. 16, the cytolysis(indicated by arrows) of epidermal cells was not observed in the peels of Jedae mandarin, unlike the peels of the mature fruits of C. unshiu Marc. cv. Miyagawa-wase, which suggests that the cytolysis of peel epidermal cells was delayed in Jedae mandarin.

The longitudinally raised portion of Jedae mandarin fruit peels were distributed more in vascular bundle, compared to the flat portion of the peels. Meanwhile, the flat portion of the peels was found to contain more intercellular spaces with cell destruction, compared to the raised portion of the peel.

Overall, fruits of mutant citrus bred in accordance with the Embodiment 1 and those of C. unshiu Marc. cv. Miyagawa-wase can be harvested almost around the same time. However, the mutant citrus has altered fruit shape(uneven peel) and inherits genetic traits same as those of mother plant in connection with the characteristics of changes in flavonoid component and therefore can be mass-produced, unlike C. unshiu Marc. cv. Miyagawa-wase. In addition, the mutant citrus can be asexually propagated with grafting. In that sense, the mutant citrus, a new variety having all characteristics described above, was named ‘Jedae mandarin’.

While the present invention was described above in detail in connection with desirable exemplary embodiments, it is to be understood that the present invention is not limited to the disclosed embodiments, but on the contrary, is intended to encompass various modifications and improvements to be made by those skilled in relevant field of technology, which are defined within the spirit and scope of the appended claims as follows:

1. A variant of citrus plant of a novel variety named ‘Jedae mandarin’, substantially as illustrated and described herein.

2.-4. (canceled)
